(19) United States
(12) Patent Application Publication
(10) Pub. No.: US 2002/0007220 A1
(43) Pub. Date: Jan. 17, 2002

(54) FEMORAL COMPONENT FOR USE IN REPLACEMENT HIP JOINT

(76) Inventors: GRAHAM ALLAN GIE, DEVON (GB); ROBIN SYDNEY MACKWOOD LING, DEVON (GB); JOHN ANDREW STORER, BAYEUX (FR); ANDREW JOHN TIMPERLEY, EXETER (GB)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 09/464,833

(22) Filed: Dec. 16, 1999

(30) Foreign Application Priority Data
Dec. 21, 1998 (GB) 9828210.6
Dec. 21, 1998 (GB) 9828212.2

(51) Int. Cl. A61F 2/36

(52) U.S. Cl. 623/23.15; 623/23.18; 623/23.35

(57) ABSTRACT

A femoral component for a hip replacement joint having a tapered collarless stem for being cemented in a medullary canal. The proximal portion of the stem has a taper and the distal segment is generally circular in cross-section and has a length equal to or greater than the proximal portion. The component can be made in two separate pieces and can be coupled in the metaphysical area of the femur. In one embodiment, at the proximal end of the stem, there is a pronounced laterally projecting heel adapted to extend into the greater trochanter of the femur of the femur into which it is to be filled. Thus, the distal facing part of the heel can provide a lateral portion which is greater than that of known collarless stems and which is particularly adapted to resist torque. In order to fit the femoral component in the medullary canal, it is necessary to broach the medullary canal so that there is a cavity within the greater trochanter.

### BACKGROUND OF THE INVENTION

1. Field of the invention.

This invention relates to a femoral component for use in a replacement hip joint.

2. Discussion of Prior Art.

The “Exeter” type femoral component of the kind shown in British patent 1 409 054 is well known and comprises a neck which carries a ball head for cooperation with an acetabular socket. Such a stem is also shown in U.S. Pat. Nos. 3,793,650 and 5,171,275. the teachings of which are incorporated herein by reference. The neck is connected to a tapered collarless stem. Thus, there is no collar for resting either on the bone or the cement in the area where the stem joins the neck of the implant. This type of stem has evolved so that the stem can be given a polished finish to help it subside, i.e. slide down inside the hardened bone cement after implantation during use as the cement deforms gradually (creeps) and the present invention relates to this type of femoral component.

In certain cases, for example, where the medullary canal is particularly narrow, it is difficult to ensure that a stem of standard shape is firmly housed. In other cases, it may be preferable to encourage proximal load transfer to the bone.

The present invention is intended to overcome some of the above disadvantages and comprises a femoral component of a replacement hip joint which has a tapered collarless stem for fixing in a medullary canal by cement, and in which said stem has a proximal portion which has a straight or curved taper, and an extended elongated distal portion which is adapted to extend into the shaft of the bone. A stem showing a concave taper is shown in U.S. Pat. No. 5,458,651.

The extended distal portion can act to centralize the stem and the tapered surfaces act to encourage proximal load transfer to the bone. Preferably, the elongated distal portion of the stem is substantially circular in cross-section although it can be other cross-sections if desired.

This elongated distal portion can be at least the same length as said proximal portion and in a preferred embodiment is substantially twice as long as the tapered proximal portion. The taper angle of the proximal portion can be slightly steeper than in known constructions on the proximal part of the stem.

At the hip joint bearing surfaces there is a load transfer from the acetabular component into the femoral component of the implant. Beyond the distal end of the femoral component stem all this load has been transferred into the bone. Between the cut or resected end of the femur proximally and the distal tip of the femoral stem the load gradually transfers from the implant into the bone. The distribution of this load transfer along the length of the femoral stem is influenced at each cross-sectional level by the relative stiffness of the implant, the bone cement mantle and the surrounding bone. Many femoral hip stem implants have large cross-sections near their distal tip giving high sectional stiffness and this causes a high proportion of the load from the bearing surface to be retained within the implant and transferred out through the bone cement mantle into the bone near the distal end of the implant. Conversely, stems which are very flexible distally (by virtue of the choice of material modulus or sectional geometry) cause a greater proportion of the load to be transferred into the bone at or near the proximal end of the femur.

Subsidence of the stem within the cement includes an increase in stem section to be accommodated by the cement proximally leaving a residual hoop strain, thus causing proximal load transfer into the bone.

It has also been found that it is preferable to provide anchorage for the stem to resist as much as possible rotation of the stem after implantation. The present invention is also intended to provide a femoral component which takes advantage of this facility.

In known constructions of femoral components of this type, for example as shown in British patent 1 409 054, the lateral side of the component is usually straight or tapered in relation to its longitudinal axis and leads directly to a shoulder at the proximal end which curves around to meet the neck which carries the ball head. Such components are fitted by reaming out the medullary canal appropriately. There is, however, space laterally in the bone within the greater trochanter and the femoral component according to the present invention is intended to take advantage of this to provide greater resistance to stem rotation after implantation.

### SUMMARY OF THE INVENTION

The arrangement according to the invention therefore provides relative rigidity at the top end of the implant which is greater than the distal end. This transfers more less load from the hip stem onto the bone through the bone cement mantle at the top and less passes down the stem and is transferred out into the bone at the distal end. Transfer of load onto the top end of the femur is thought to be beneficial in order to avoid bone resorbtion.

A further advantage is that at the present in some patients a point about half way up the stem in existing devices is found to come very close to the internal bone wall and leaves very little space for cement. Thus, although the existing type of stem has straight tapering edges, the inside form of the cortical bone is more trumpet shaped. Therefore, by providing concave surfaces on the stem, the thickness of the cement mantle thickness laterally in this area can be increased to a more acceptable thickness, at least 1 mm and preferably 1mm - 2mm. It is important in the present arrangment that the edges and side faces of the stem never become parallel over the proximal part of the stem because this will lose the advantage of taper locking engagement either before or after any subsidence. The stem must be always narrowing as it progresses downwards but it is not necessary to maintain a constant taper angle.

Preferably the surface of the stem is polished and it can be used with a distal void centralizer such as that shown in U.S. Pat. No. 5,092,892.

In the embodiments referred to above, the proximal part of the femoral component and the distal stem are in one piece but, in an alternative construction, the proximal portion is provided with means for connection to a separate elongated distal portion.

Because it is intended that the elongated distal portion should not carry any of the load, it can be provided by what is in effect an elongated centralizer, and this can be made from metal or, for example, from a synthetic plastics material such as polymethylmethacrylate. The elongated distal portion can be solid or hollow and, if desired, it can be provided with a centralizer element in the form of flanges or other projections.

According to the present invention, a femoral component of a replacement hip joint which has a tapered collarless stem for fixing in a medullary canal by bone may have an embodiment where at the proximal end of the stem, there may be a pronounced laterally projecting heel adapted to extend into the greater trochanter of the femur into which it is to be fitted. Thus, the distal facing part of the heel can provide a lateral portion which is greater than that of known collarless stems and which is particularly adapted to resist torque. It will be appreciated that, in order to fit the femoral component according to the invention, it is necessary to broach the medullary canal so that there is a cavity within the greater trochanter.

### BRIEF DESCRIPTION OF THE DRAWINGS

The invention can be performed in various ways and two embodiments will now be described by way of example and with reference to the accompanying drawings, which:

FIG. 1 is a side elevation of a femoral component according to the invention located in a femur;

FIG. 2 is a front elevation of the component shown in FIG. 1;

FIG. 3 is a cross-sectional end view on the line III-III of FIG. 2; and

FIGS. 4 and 5 are similar views to FIGS. 1 and 2 but of an alternative construction.

FIG. 6 is a side view of a femoral component according to one embodiment the invention shown in position in a femur; and

FIG. 7 is an end view of the device shown in FIG. 6.

### DESCRIPTION OF THE PREFERRED EMBODIMENTS

As shown in FIGS. 1 to 3 of the drawings, the femoral component of a replacement hip joint has a collarless stem 1 of substantially rectangular cross-section. The stem 1 is intended for fixing in position in a medullary canal 2 of a femur indicated by broken lines 3 by bone cement in a well known manner. The proximal portion of the stem has a continuous taper from a point indicated by broken line 4 to a proximal portion 5 where it merges into a neck 6. The neck 6 communicates with a boss 7 to receive a ball head indicated by broken lines 8 which will cooperate with an acetabular socket.

The anterior side face 9 of the stem including portion 5 is substantially flat until it merges into the neck 6 which is of circular cross-section. The posterior side face 10 is of similar configuration. These faces are radiussed with a longitudinally extending curve so that they are concave. This shape extends from the line 4 and up through portion 5 on these faces to neck 6. At the upper end there is a high concave radius as indicated by reference numeral 11, the radius decreasing thereafter and finally running out at the point 4. The center line of the tapering straight stem portion is indicated at 14.

The lateral face 15 and the medial face 16 of the stem are also tapered below the portion 5. The curving inner medial face 17 of the portion 5 is of a greater angle to the axis 14 than normal and merges into a straight cylindrical portion of the stem at the point where it intersects line 4 and the concave surface 19 on the lateral face of portion 5 is also at a greater angle and merges into a straight portion of the stem at the same point.

Below point 4 the stem is in the form of an elongated cylindrical distal portion 20. Below portion 5 the medial faces 15 and 16 are also shaped concave down to the point 4.

As shown in FIG. 1, the faces 17 and 19 are curved but they could be straight.

As shown in the drawings the elongated distal portion 20 is at least the same length as the tapered proximal portion above the line 4 and which is indicated by reference numeral 21. Preferably, and as shown in the drawings, the elongated distal portion 20 is substantially twice as long as the tapered proximal portion 21.

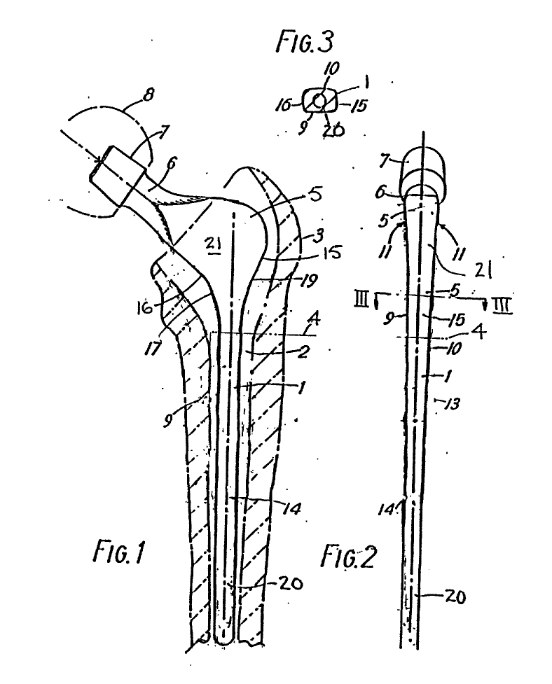

The cross-section of the extended portion 20 can be any convenient shape but, as shown in FIG. 3, it is preferably circular.

The cross-section of the stem above the line 4 is as shown in FIG. 3 and is substantially rectangular with radiussed comers. The surfaces can be flat but in the arrangement shown they are slightly bowed outwardly. In the arrangement shown the femoral component is intended for use with a removable ball head 8 but if desired the ball head could be integral with the stem.

The surface of the stem incorporating the portion 5 is highly polished and if desired a distal void centralizer (not shown) for example of the kind set forth in British Patent 2 104 391 or U.S. Pat. No. 5,092,892 can be used with it.

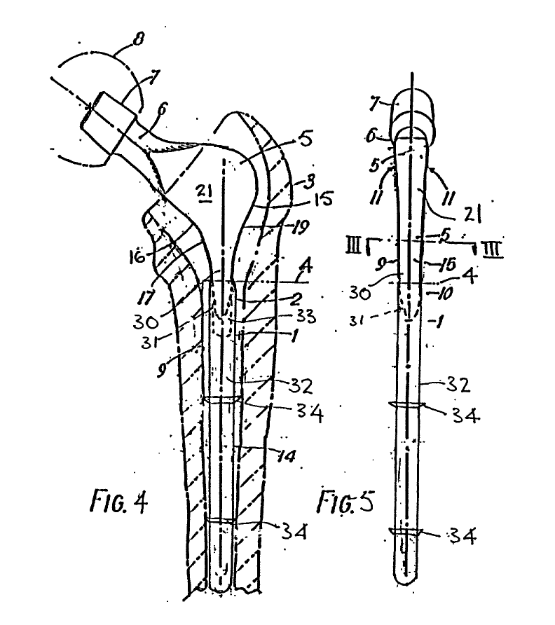

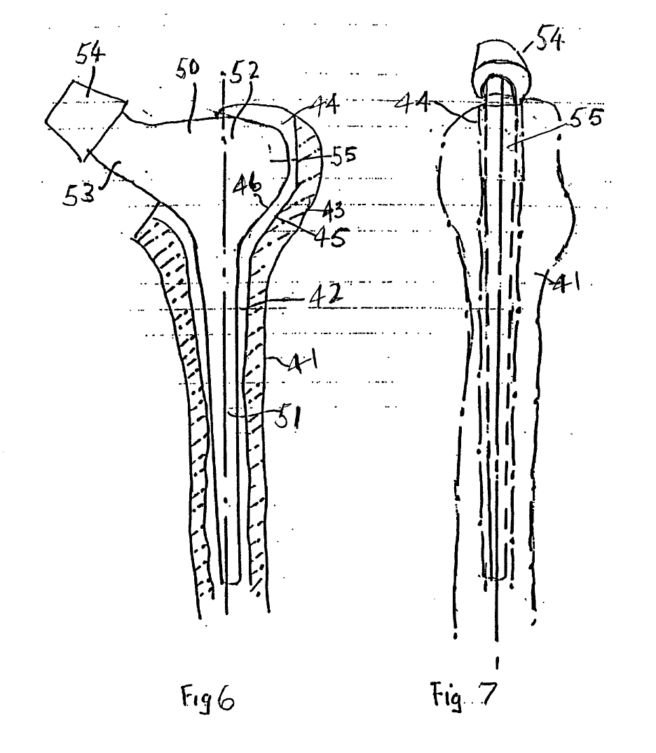

In the construction described above, the proximal portion and elongated distal portion 20 are integral and made as a single element but, in the arrangement shown in FIGS. 4 and 5, the two portions are made separately, but the same reference numerals are used to indicate similar parts.

As shown in FIGS. 4 and 5, the tapered proximal portion 21 is made from metal and has a short tail or tip 30, the distal end of which is tapered as shown at 31. An elongated distal portion 32 has a hollow proximal end 33 which is a tight push fit over the tapered portion 31. The distal portion 32 can be made from metal or a plastics material and it can be provided with centralizer flanges or abutments 34. If desired, the distal portion 32 can be hollow over the whole of its length or substantially solid as shown in the drawings. Its outer surface is smooth apart from the centralizer flanges 34 and the whole femoral component sink into the cement in known fashion.

The distal portion 32 can be relatively flexible because the main loading on the component is carried beneath the proximal shoulder. The proximal portion 33 of the distal section can have a hollow pocket below the tip of distal end 31, which is deeper than the length of the tip of the proximal portion, to allow for the subsidence of end 31 of portion 21 distally in a manner similar to that discussed in U.S. Pat. No. 5,092,892. This is especially important if a plastic distal stem portion is utilized.

As shown in FIGS. 6 and 7, a cross-sectioned femur is indicated by reference numeral 41. The intramedullary canal 42 of the femur has been broached to accept a femoral component, and it will be seen that, at the greater trochanter indicated by reference numeral 43, the canal 42 has been opened out in a lateral direction which caused an exposed portion of the canal 44 to extend as a slot within the greater trochanter 43.

The proximal part of the prepared canal has a pronounced angle portion 45.

The especially adapted femoral component for use in such a canal is indicated by reference numeral 50 and comprises a stem 51, a proximal portion 52 and a neck 53 on which is located a spigot or trunion 54 to accept a ball head (not shown). The stem is collarless and either straight or tapered along the medial posterior and anterior sides. On the lateral side, the distal stem is also either straight or tapered until meeting transition surface 56.

The proximal end of the stem, where it leads into the proximal portion 52, has a pronounced laterally projecting heel 55 which extends into the greater trochanter 43 when it is fitted. The head may extend to within several millimeters of the cortical bone in the trochanteric area. As will be seen from FIG. 6, the particular widely-splayed shape of the proximal portion 52 provides a distal surface 56 to the heel, which surface can be concave, convex or straight, but increased the lateral width of the component to provide not only a greater area of contact to absorb loads and resist torsional displacement, but to absorb distal loads in general. In the preferred embodiment, the shapes of surface 45 and 46 are complimentary and are several millimeters apart.

The invention has great advantages over components of standard shape and can be fitted in most patients, provided there is sufficient healthy bone.

1. A femoral component for a replacement hip joint having a tapered collarless stem for fixing in a medullary canal by cement, and in which said stem has a proximal portion which has a straight or curved taper, and an extended elongated distal portion which is adapted to extend into the shaft of the bone.

2. The femoral component as claimed in claim 1 in which the elongated distal portion of the stem is substantially circular in cross-section.

3. The femoral component as claimed in claim 1 in which said elongated distal portion is at least the same length as said tapered proximal portion.

4. The femoral component as claimed in claim 3 in which said elongated distal portion is substantially twice as long as said tapered proximal portion.

5. The femoral component as claimed in claim 1 in which none of the edges and side faces of the proximal part of said stem are parallel.

6. The femoral component as claimed in claim 1 in which the surface of the stem is polished.

7. The femoral component as claimed in claim 1 in which the proximal portion of the component and the stem are in one piece.

8. The femoral component as claimed in claim 1 in which the proximal portion of the component is provided with means for connection to a separate elongated distal portion.

9. The femoral component as claimed in claim 8 in which said elongated distal portion is adapted to effectively act as an elongated centralizer.

10. The femoral component as claimed in claim 9 in which said elongated distal portion is made of metal or a synthetic plastics material.

11. The femoral component as claimed in claim 1 in which said elongated distal portion is hollow.

12. The femoral component as claimed in claim 1 in which said elongated distal portion is provided with a centralizer.

13. The femoral component as claimed in claim 12 in which said centralizing means are in the form of flanges or other projections.

14. A femoral component for a hip replacement joint comprising:
a distal stem portion;
a collarless proximal portion having or generally rectangular cross-section, said cross-section tapering inwardly along all sides upon moving from proximal to distal, said proximal portion including a distal tapered portion for insertion into said pocket of said stem portion.

15. The femoral component as set forth in claim 14, wherein the distal stem portion has a generally circular cross-section.

16. The femoral component as set forth in claim 15 wherein the proximal portion is polished.

17. The femoral component as set forth in claim 14 wherein the distal stem portion is made of plastic.

18. The femoral component as set forth in claim 17 wherein said pocket has a depth greater than the length of the distal tapered portion of the proximal portion.

19. The femoral component as set forth in claim 14 wherein the inward taper is curved.

20. The femoral component as set forth in claim 14 wherein said distal portion includes centralizers attached thereto.

21. A femoral component for a replacement hip joint comprising a tapered collarless stem for fixing in a medullary canal by cement and in which, at the proximal end of the stem, there is a pronounced laterally projecting heel adapted to extend into the greater trochanter of the femur into which it is to be fitted.

22. The femoral component as claimed in claim 21 in which the distal surface of said heel is substantially concave.

23. The femoral component as claimed in claim 21 in which the distal surface of said heel is substantially convex.

24. The femoral component as claimed in claim 21 in which the distal surface of said heel is substantially straight.

25. A femoral component for implantation into a prepared femoral cavity comprising:
a collarless proximal portion having a generally rectangular cross-section with tapered medial, anterior and posterior surfaces and having an enlarged proximal portion in the lateral direction, said enlarged proximal portion extending laterally to within several millimeters of cortical bone in a trochanteric area of the femoral cavity.

26. The femoral component as set forth in claim 25 wherein said enlarged proximal portion has a tapered distal surface extending to within several millimeters of said trochanter region and complimentary thereto.
